(19) United States
(12) Patent Application Publication
(10) Pub. No.: US 2004/0132655 A1
(43) Pub. Date: Jul. 8, 2004

(54) New combination

(76) Inventor: Philip Harris, Blackheath (GB)

Correspondence Address: DINSMORE & SHOHL, LLP, 1900 CHEMED CENTER, 255 EAST FIFTH STREET, CINCINNATI, OH, 45202, US

(21) Appl. No.: 10/677,931

(22) Filed: Oct. 2, 2003

Related U.S. Application Data
(60) Provisional application No. 60/415,666, filed on Oct. 3, 2002.

(51) Int. Cl. A61K 38/27; A61K 31/415

(52) U.S. Cl. 514/12; 514/406

(57) ABSTRACT

A combination of a growth hormone receptor antagonist and a COX-2 inhibitor is useful for treating headaches in patients with acromegaly.

### RELATED APPLICATIONS

This application claims priority under 35 U.S.C. §119 of Application Serial No. 60/415,666 filed Oct. 3, 2002.

### INTRODUCTION

A combination of a growth hormone receptor antagonist and a COX-2 inhibitor is useful for treating headaches in patients with acromegaly.

### BACKGROUND OF THE INVENTION

A growth hormone receptor antagonist blocks the growth hormone receptor and inhibits growth hormone to bind with the said receptor to induce the biological effect. In patients with acromegaly, excess amount of growth hormone are produced in the body due to many pathological causes. The said growth hormone antagonists will benefit the said patients to treat acromegaly.

Acromegaly is frequently associated with severe headaches. Some of patients response to standard analgesics. Somatostatin analogues have been used to treat headaches of the patients with acromegaly (Musolino N R. Marino Junior R. Bronstein M D., Clinical Journal of Pain. 6(3):243-5, 1990 September). However there are a proportion of patients whose headaches remain refractory to a conventional treatment, e.g. Somatostatin analogues. Somatostatin analogues inhibit the secretion of growth hormone and do not affect the growth hormone receptor. Somatostatin analogues may be used by their direct pharmacological effect on pain pathways for the headaches (Popovic, V. et al, Horm. Metabol. Res. 20, pp 250-251, 1988), rather than a secondary effect on growth hormone levels or tumor size that often cause severe headaches in the patients. Somatostatin analogues have some side effects that may limit their use in some of the patients (Freda P. U., Journal of Clinical Endocrinology & Metabolism. Vol 87(7) pp 3013-3018, 2002).

Pegvisomant (Somavert®) is a growth hormone receptor antagonist which is effective in the treatment of acromegaly (WO 97/11178). It improves the clinical symptoms of acromegaly and normalizes insulin-like growth factor-1-(IGF-1). But headaches in the patients with acromegaly have not got effective relief in the course of the treatment.

COX-2 inhibitors are a class of drug that selectively inhibits cyclo-oxygenase-2 (COX-2), also known as PGH synthase or prostaglandin endoperoxide synthase. They are used as non-steroidal anti-inflammatory drugs for arthritis, pain and other COX-2 related disorders (Cannon G. W., Cyclooxygenase-2 selective inhibitors. Drugs of Today. Vol 35(7) pp 487-496, 1999).

Celecoxib is a COX-2 inhibitor used as a non-steroidal anti-inflammatory drug with a highly selective cyclo-oxygenase-2 (COX-2) inhibitory action. It has anti-inflammatory, analgesic, and antipyretic activities (WO 95/15316).

### SUMMARY OF THE INVENTION

This invention discloses a therapeutic method comprising the combination of a growth hormone antagonist and a COX-2 inhibitor for treating headaches in patients with acromegaly by administration of the said combination to the patients in need of such treatment. More precisely, the invention relates to the use of the combination of pegvisomant and a COX-2 inhibitor for treatment of a patient with headache associated with acromegaly wherein the combination comprising therapeutically effective amount and pharmaceutically acceptable dosage forms or delivery systems of the said growth hormone receptor antagonists and COX-2 inhibitors.

### DETAIL DESCRIPTION OF THE INVENTION

The present invention is related to a combination therapy for a patient with headaches associated with acromegaly. The combination comprises a growth hormone receptor antagonist and a COX-2 inhibitor in a therapeutically effective amount and pharmaceutically acceptable dosage forms and delivery systems.

A growth hormone receptor antagonist, as it is used herein, is a substance that binds growth hormone receptors and does not induce biological action of the said receptors. It may be competitive or non-competitive with growth hormone to bind the said receptors. A growth hormone receptor antagonist can be selected from, but not limited to, a human growth hormone variant, its pegylated form, or its pro-drugs thereof. A preferred growth hormone receptor antagonist is pegvisomant with a possible dose range from 1 μg/kg/day to about 100 mg/kg/day of patient body weight.

A COX-2 inhibitor, as it is used herein, is a substance that selectively binds with cyclo-oxygenase-2 and inhibits the function of the said enzyme. It can be competitive or non-competitive with the native substrates to the said enzyme. According to the present invention, the COX-2 inhibitor can be selected from, but not limited, e.g. celecoxib, valdecoxib, parecoxib, rofecoxib, etoricoxib, iguratimod, lumiracoxib, tilmacoxib. Preferred COX-2 inhibitor for the combination is celecoxib or valdecoxib or parecoxib in a dose range of 0.1 to 2000 mg, preferably in the range of 0.5 to 500 mg and most preferably between about 1 and 100 mg. A daily dose of about 0.01 to 100 mg/kg body weight, preferably between about 0.1 and about 50 mg/kg body weight and most preferably from about 1 to 20 mg/kg body weight, may be appropritate.

According to the invention, by using the method that comprises the administration of the combination of a growth hormone receptor antagonist and a COX-2 inhibitor, the headaches in patients with acromegaly have surprisingly been found improved or cured.

According to the invention, the growth hormone receptor antagonist and the COX-2 inhibitor are preferably administrated in a pharmaceutical acceptable form. The administration of each component in the said combination may take place separately, simultaneously or sequentially by any conventional route, e.g. parenterally, e.g. in the form of injectable solutions or suspensions, orally, e.g. in the form of drink solutions, emulsions, microemulsions, tablets, capsules, pulmonary, e.g. inhalation forms, nasally or topically.

### EXAMPLE

6 acromegalic patients with normal IGF-1 (Insulin-like Growth Factor-1) levels on the Somavert® treatment, who continue to have headaches, are treated with the said combination in a double blind cross-over fashion with a COX-2 inhibitor (celecoxib 100 mg bd for 2 weeks, increasing to 200 mg bd if required) or placebo for 4 weeks each, with one week washout. Visual analogue score (0-10) for headache on weekly basis are measured and recorded for all patients in the experiment.

1. A method for treating headaches in a patient with acromegaly comprising administration of a growth hormone receptor antagonist and a COX-2 inhibitor to the said patient in need of the treatment.

2. The method according to claim 1, wherein the human growth hormone antagonist is selected from a human growth hormone variant, its pegylated form, or its pro-drug form.

3. The method according to claim 2, wherein the human growth hormone antagonist is pegvisomant.

4. The method according to claim 1, wherein the COX-2 inhibitor is selected from celecoxib, valdecoxib, parecoxib, rofecoxib and etoricoxib.

5. The method according to claim 2, wherein the COX-2 inhibitor is selected from celecoxib, valdecoxib, parecoxib, rofecoxib and etoricoxib.

6. The method according to claim 3, wherein the COX-2 inhibitor is selected from celecoxib, valdecoxib, parecoxib, rofecoxib and etoricoxib.

7. The method according to claim 4, wherein the COX-2 inhibitor is celecoxib or valdecoxib.

8. A pharmaceutical composition comprising a pharmaceutical acceptable carrier and a therapeutically effective amount of a growth hormone receptor antagonist and a COX-2 inhibitor.

9. A method for preparation of a medicament comprising a growth hormone receptor antagonist and a COX-2 inhibitor for the treatment of headaches in a patient with acromegaly.
